(19) United States
(12) Patent Application Publication
(10) Pub. No.: US 2022/0074882 A1
(43) Pub. Date: Mar. 10, 2022

(54) ELECTROCHEMICAL-BASED SENSOR FOR RAPID AND DIRECT DETECTION OF SARS-COV-2

(71) Applicant: FRAUNHOFER USA, INC., Plymouth, MI (US)

(72) Inventors: Suzanne WITT, Plymouth, MI (US); Michael BECKER, Plymouth, MI (US)

(21) Appl. No.: 17/466,998

(22) Filed: Sep. 3, 2021

Related U.S. Application Data
(60) Provisional application No. 63/074,914, filed on Sep. 4, 2020.
(60) Provisional application No. 63/121,028, filed on Dec. 3, 2020.

(51) Int. Cl. G01N 27/30; G01N 33/569

(52) U.S. Cl. CPC G01N 27/308; G01N 2800/26; G01N 27/301; G01N 33/56983

(57) ABSTRACT

Disclosed herein are electrochemical-based sensors, comprising: a solid electrode material; a linker moiety bound to the solid electrode material; and a receptor bound to the linker moiety, wherein the receptor binds to a target, and the binding of target to receptor causes an increase in the charge transfer resistance of the solid electrode material. In particular, the present disclosure relates to an electrochemical sensor which is selective for the S₁ subunit of the SARS-CoV-2 spike protein and which uses boron-doped diamond as a solid electrode material. Sensor networks comprising one or more such sensors are also disclosed herein, along with methods of detecting a target (e.g., SARS-CoV-2) using such sensors.

### CROSS-REFERNCE TO RELATED APPLICATIONS

This application claims the benefit of and priority to U.S. Provisional Patent Application No. 63/074,914, filed Sep. 4, 2020, and U.S. Provisional Patent Application No. 63/121,028, filed Dec. 3, 2020, the entire disclosures of which are hereby incorporated by reference herein.

### BACKGROUND

The following discussion is merely provided to aid the reader in understanding the disclosure and is not admitted to describe or constitute prior art thereto.

Rapid detection of pathogens is crucial to tracking the potential spread of disease and to mitigating and/or preventing public health crises stemming from the same. For example, coronavirus disease 2019 (COVID-19) (also referred to as novel coronavirus pneumonia or 2019-nCoV acute respiratory disease) is an infectious disease caused by the virus severe respiratory syndrome coronavirus 2 (SARS-CoV-2) (also referred to as novel coronavirus 2019, or 2019-nCoV). The disease was first identified in December 2019 and spread globally, causing a pandemic. Symptoms of COVID-19 include fever, cough, shortness of breath, fatigue, headache, loss of smell, nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, and kidney failure. Complications can include pneumonia, viral sepsis, acute respiratory distress syndrome, and kidney failure.

COVID-19 is especially threatening to public health. The virus is highly contagious, and studies currently indicate that it can be spread by asymptomatic carriers or by those who are pre-symptomatic. Likewise, the early stage of the disease is slow-progressing enough that carriers do not often realize they are infected, leading them to expose numerous others to the virus. The combination of COVID-19's ease of transmission, its high rate of hospitalization of victims, and its death rate make the virus a substantial public health risk, especially for countries without a healthcare system equipped to provide supportive care to pandemic-level numbers of patients. And although vaccines and/or treatments have become widely available since the onset of the pandemic, there remains a substantial commercial interest in rapid, selective, and sensitive means for detecting COVID-19 to assist in tracking and containing outbreaks.

SARS-CoV-2 is not the only coronavirus that causes disease. It is a β-coronavirus, a genus of coronaviruses that includes other human pathogens, including SARS-CoV (the causative agent of SARS), MERS-CoV (the causative agent of MERS), and HCoV-OC43 (a causative agent of the common cold). The infectivity of these viruses, and the severity of the diseases they cause, varies widely. β-coronaviruses can also manifest as zoonotic infections, spread to and from humans and animals. Additionally, non-human species such as camels, bats, tigers, non-human primates, and rabbits can be susceptible to β-coronaviruses. Accordingly, there is a pressing need for systems and sensors for detecting to multiple coronaviruses and, in particular, SARS-CoV-2.

### SUMMARY

Boron Doped Diamond (“BDD”) exhibits the widest electrochemical potential window of all solid electrode materials and is known to be resistant to fouling, highly customizable, and readily functionalized to facilitate sensitivity to many different microbial targets (e.g., viruses and bacteria).

The present technology is not intended to be limited to detection of any particular pathogen, and electrochemical sensors disclosed herein may be used to detect any pathogen (e.g., any emerging pathogen), particularly those that pose a high risk to public health. For example, no BDD-based electrochemical sensors have been developed for specific detection of SARS-CoV-2 across a variety of sample media or surfaces. There exists great commercial interest in non-reagent electrochemical detection technology that is sensitive, able to analyze various media (e.g., saliva droplets and aerosols, solid surfaces, ambient air, water and wastewater) and digitally-enabled for data capture and dissemination. Thus, there is immediate interest in an electrochemical biosensor for SARS-CoV-2 that can offer a rapid, digital, highly selective, and highly sensitive alternative to conventional detection methods (e.g., PCR, plating, or similar methods), which are costly, slow, and inaccurate. Although the example embodiment recites the detection of SARS-CoV-2, it is intended that the systems and methods can be configured for any pathogen.

In one aspect, the present disclosure relates to an electrochemical-based sensor, comprising: a solid electrode material; a linker moiety bound to the solid electrode material; and a receptor bound to the linker moiety, wherein the receptor binds to target, and the binding of target to receptor causes an increase in the charge transfer resistance of the functionalized sensor.

In some embodiments according to the present disclosure, the solid electrode material comprises boron-doped diamond (“BDD”). In some embodiments, the linker moiety comprises a biotin-streptavidin complex. In some embodiments, the linker moiety comprises a biotin-streptavidin complex.

In some embodiments, the receptor comprises an antibody. The antibody can be specific for any pathogen (e.g., emerging pathogens that pose a risk to public health) or for any protein derived from any such pathogen. In some embodiments, the receptor comprises an antibody specific for SARS-CoV-2. In some embodiments, the antibody is biotinylated. In some embodiments, the antibody is specific for a spike protein of SARS-CoV-2. In some embodiments, the antibody is SARS-CoV-2 (2019-nCoV) Spike S₁ Antibody, Rabbit MAb (Sino Biological; Cat: 40150-R007). In some embodiments, the antibody is specific for a S₁ subunit of the spike protein. In some embodiments, the antibody is specific for a S₂ subunit of the spike protein. In some embodiments, the target comprises SARS-CoV-2 (e.g., live SARS CoV-2).

In some embodiments according to the present disclosure, the sensor further comprises a housing enclosing the solid electrode material, linker moiety, and receptor. In some embodiments, the sensor is reusable.

In another aspect, the present disclosure relates to a network of biomolecule sensors, comprising: a first sensor according to any of the above embodiments, wherein the first sensor is placed at a first location; a second sensor according to any of the above embodiments, wherein the second sensor is placed at a second location; and a processing unit in communication with the first sensor and the second sensor. In some embodiments, the sensor may be used on humans (e.g., in an oral device, such as a breathalyzer, or by contact with a nasal swab), used on inanimate objects (e.g., embedded in an HVAC system, duct, air filter, water supply line, wastewater line, surface, etc.), or applied in hybrid use applications (e.g. sensor integrated with a mobile phone).

In some embodiments, the first location is associated with a heating and/or ventilation system, a surface (e.g., a contaminated surface), a breath-capture device (e.g., a breathalyzer), a water supply system, wastewater system, saliva capture device, or nasal swab. In some embodiments, the second location is associated with a heating and/or ventilation system, a surface (e.g., a contaminated surface), a breath-capture device (e.g., a breathalyzer), a water supply system, wastewater system, saliva capture device, or nasal swab. In some embodiments, the first sensor and the second sensor are specific for the same target (e.g., SARS-CoV-2). In some embodiments, the output digital fingerprint caused by the binding of a target (e.g., SARS-CoV-2) by each sensor in a specific medium may be unique.

In another aspect, which may be combined with any other aspect or embodiment, the present disclosure relates to a method of detecting a target in a medium, the method comprising: (a) contacting a sensor according to any of the above-discussed embodiments with a medium containing or suspected of containing a target, wherein the contacting is performed over a time period during which the target present within the medium binds to one or more receptors on the sensor; (b) measuring a property of the sensor after the target has bound to the one or more receptors; (c) comparing the property of the sensor after the target has bound to the one or more receptors to the property of the sensor before the contacting to determine a change in the property of the sensor; and (d) determining the amount of target present in the medium based on the change in the property of the sensor. In some embodiments, the method is a method of detecting SARS-CoV-2 in a medium.

In some embodiments, the target comprises SARS-CoV-2. In some embodiments, the property is charge transfer resistance. In some embodiments, the sensor comprises a solid electrode material comprising boron-doped diamond (BDD). In some embodiments, the medium comprises at least one of: ambient air; exhalation by a human or animal subject; a liquid comprising a physiological fluid; a liquid from a municipal water source, or any combination thereof.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration of the general surface functionalization steps that may be used to produce an electrochemical sensor according to the present disclosure.

FIG. 2 is a schematic illustration of the surface functionalization steps used to produce an electrochemical SARS-CoV-2 sensor, according to one embodiment of the present disclosure.

FIG. 3 shows photographs of an electrochemical cell (left) and a 3-in-1-sensor (right) incorporating the electrochemical SARS-CoV-2 sensor produced according the present disclosure.

FIG. 4 is a circuit diagram for an electrochemical SARS-CoV-2 sensor produced according to the present disclosure.

FIG. 5A shows electrochemical impedance spectra of the electrochemical SARS-CoV-2 sensor produced according to the present disclosure, after binding of the S₁ subunit of the SARS-CoV-2 spike protein from solutions containing different concentrations of the spike protein.

FIG. 5B shows charge transfer resistance (R_{ct}) extracted from fitted EIS spectra as a function of S₁ subunit concentration.

FIG. 6 is a plot of normalized R_{ct} values as a function of protein concentration for the SARS-CoV-2 spike protein subunit (orange) and for the Influenza B hemagglutinin protein (blue). (Error bars indicate one standard deviation.)

FIG. 7 shows normalized R_{ct} values over a range of SARS-CoV-2 concentrations (x) compared to 1 μL additions of cell culture medium only (●). The SARS-CoV-2 concentration for each addition is denoted above the points in the plot. The data points denote different incubation times, progressing from smallest to largest (small: 30 s; medium: 2 min; large: 5 min).

FIG. 8 shows SEM images of BDD films grown on silicon wafers at different thicknesses (A: top-down, 0.7 μm; B: top-down, 3.4 μm; C: cross-section, 3.4 μm; D: cross-section, 0.7 μm).

FIG. 9 shows AFM images of BDD films having a thickness of 3.4 μm (A, left) and 0.7 μm (B, right).

FIG. 10 shows bar plots illustrating normalized R_{ct} versus antigen concentration for sensors comprising 3.4-μm BDD films (A, left) and 0.7-μm BDD films (B, right) tested against SARS-CoV-2 S₁ subunit (red) and Influenza B Hemagglutinin protein (blue). The plotted data represents the average of three total sensors; error bars represent one standard deviation.

FIG. 11 shows a system architecture, according to an embodiment.

### DETAILED DESCRIPTION

The electrochemical-based sensors according to the present disclosure may comprise an electrically-conductive substrate (solid electrode) functionalized with one or more linker moieties and one or more receptors, which together are capable of rapidly and specifically binding one or more targets (e.g., analytes or biomolecules, including SARS-CoV-2 and/or biological materials directly related to or derived from SARS-CoV-2).

Solid Electrode

In some embodiments, the electrochemical-based sensor of the present disclosure comprises a solid electrode material. The solid electrode material may be any suitable material for use as an electrode (e.g., is conductive) and which is capable of being functionalized with one or more types of receptor molecules. In some embodiments, the solid electrode material may comprise diamond or diamond-like materials (e.g., single-crystal diamond (“SCD”), nanocrystalline diamond (“NCD”), microcrystalline diamond (“MCD”), etc.), other carbon-based materials (e.g., graphene), one or more metals (e.g., Au, Ag, Cu, Pt, etc.), conductive oxides (e.g., ITO), or semiconductor materials (e.g., Si), or any other suitable material. In some embodiments, the solid electrode material may comprise SP2 materials, SP3 materials, or mixtures thereof. In some embodiments, the solid electrode material is boron-doped diamond (“BDD”).

In some embodiments, the solid electrolyte material may be deposited on a base substrate (e.g., conductive Si, SiO₂, or metals including Nb, W, Mo, Ta, etc.). In some embodiments, the solid electrolyte material may be deposited onto a base substrate by any suitable method (e.g., physical vapor deposition, chemical vapor deposition, electrochemical methods, etc.).

The solid electrode material (e.g., BDD) may be deposited on a substrate in any suitable thickness for achieving high specificity and sensitivity of electrochemical sensors prepared therefrom. In some embodiments, the solid electrode material may have a thickness of at least about 0.01 μm, at least about 0.02 μm, at least about 0.03 μm, at least about 0.04 μm, at least about 0.05 μm, at least about 0.06 μm, at least about 0.07 μm, at least about 0.08 μm, at least about 0.09 μm, at least about 0.1 μm, at least about 0.2 μm, at least about 0.3 μm, at least about 0.4 μm, at least about 0.5 μm, at least about 0.6 μm, at least about 0.7 μm, at least about 0.8 μm, at least about 0.9 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, or any range or value therein.

In some embodiments, the solid electrode material may have a thickness of no greater than about 10 μm, no greater than about 9 μm, no greater than about 8 μm, no greater than about 7 μm, no greater than about 6 μm, no greater than about 5 μm, no greater than about 4 μm, no greater than about 3 μm, no greater than about 2 μm, no greater than about 1 μm, no greater than about 0.9 μm, no greater than about 0.8 μm, no greater than about 0.8 μm, no greater than about 0.7 μm, no greater than about 0.6 μm, no greater than about 0.5 μm, no greater than about 0.4 μm, no greater than about 0.3 μm, no greater than about 0.2 μm, no greater than about 0.1 μm, no greater than about 0.09 μm, no greater than about 0.08 μm, no greater than about 0.07 μm, no greater than about 0.06 μm, no greater than about 0.05 μm, no greater than about 0.04 μm, no greater than about 0.03 μm, no greater than about 0.02 μm, no greater than about 0.01 μm, or any range or value therein.

In some embodiments, the solid electrode material may have a thickness of about 0.01 μm, about 0.015 μm, about 0.02 μm, about 0.025 μm, about 0.03 μm, about 0.035 μm, about 0.04 μm, about 0.045 μm, about 0.05 μm, about 0.055 μm, about 0.06 μm, about 0.065 μm, about 0.07 μm, about 0.075 μm, about 0.08 μm, about 0.085 μm, about 0.09 μm, about 0.095 μm, about 0.1 μm, about 0.15 μm, about 0.2 μm, about 0.25 μm, about 0.3 μm, about 0.35 μm, about 0.4 μm, about 0.45 μm, about 0.5 μm, about 0.55 μm, about 0.6 μm, about 0.65 μm, about 0.7 μm, about 0.75 μm, about 0.8 μm, about 0.85 μm, about 0.9 μm, about 0.95 μm, about 1 μm, about 1.5 μm, about 2 μm, about 2.5 μm, about 3 μm, about 3.5 μm, about 4 μm, about 4.5 μm, about 5 μm, about 5.5 μm, about 6 μm, about 6.5 μm, about 7 μm, about 7.5 μm, about 8 μm, about 8.5 μm, about 9 μm, about 9.5 μm, about 10 μm, or any range or value therein between.

The solid electrode material (e.g., BDD) may be deposited on a substrate with any suitable surface roughness for achieving high specificity and sensitivity of electrochemical sensors prepared therefrom. In some embodiments, the surface roughness is measured using atomic force microscopy (AFM), e.g., as presented in the corresponding discussion in the Examples. In some embodiments, the solid electrode material may have a surface roughness, measured as root mean square height (S_{q}) of less than or equal to about 1 nm, less than or equal to about 2 nm, less than or equal to about 3 nm, less than or equal to about 4 nm, less than or equal to about 5 nm, less than or equal to about 6 nm, less than or equal to about 7 nm, less than or equal to about 8 nm, less than or equal to about 9 nm, less than or equal to about 10 nm, less than or equal to about 15 nm, less than or equal to about 20 nm, less than or equal to about 25 nm, less than or equal to about 30 nm, less than or equal to about 35 nm, less than or equal to about 40 nm, less than or equal to about 45 nm, less than or equal to about 50 nm, less than or equal to about 55 nm, less than or equal to about 60 nm, less than or equal to about 65 nm, less than or equal to about 70 nm, less than or equal to about 75 nm, less than or equal to about 80 nm, less than or equal to about 85 nm, less than or equal to about 90 nm, less than or equal to about 95 nm, less than or equal to about 100 nm, less than or equal to about 110 nm, less than or equal to about 120 nm, less than or equal to about 130 nm, less than or equal to about 140 nm, less than or equal to about 150 nm, less than or equal to about 160 nm, less than or equal to about 170 nm, less than or equal to about 180 nm, less than or equal to about 190 nm, less than or equal to about 200 nm, less than or equal to about 250 nm, less than or equal to about 300 nm, less than or equal to about 350 nm, less than or equal to about 400 nm, less than or equal to about 450 nm, less than or equal to about 500 nm, less than or equal to about 550 nm, less than or equal to about 600 nm, less than or equal to about 650 nm, less than or equal to about 700 nm, less than or equal to about 750 nm, less than or equal to about 800 nm, less than or equal to about 850 nm, less than or equal to about 900 nm, less than or equal to about 950 nm, less than or equal to about or any range or value therein between.

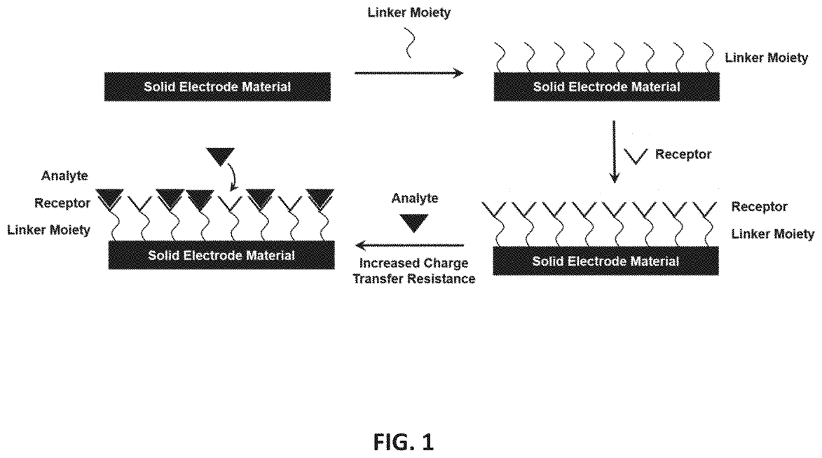

Referring to FIG. 1, in some embodiments the surface functionality of the solid electrode material may comprise any combination of common surface functional groups that may facilitate bonding of receptor molecules specific for a target (e.g., biomolecule or analyte). In some embodiments, the surface functional groups may comprise hydrogen terminal groups, hydroxyl terminal groups, carboxylic acid terminal groups, amine terminal groups, or combinations thereof. In some embodiments, the solid electrode surface functionality may be altered or tailored by one or more surface treatments (e.g., by plasma treatment, UV/ozone treatment, or wet chemical treatment). In some embodiments, the solid electrode surface functionality may be altered or tailored for specific applications by deposition of thin films onto the solid electrode material (e.g., by vapor-phase or solution deposition of self-assembled monolayers of, e.g., silane, carboxylate, phosphonate, amine, or thiol molecules with any appropriate terminal functional groups). In some embodiments, the surface of a BDD solid electrode is hydrogen terminated by treatment in hydrogen plasma, then hydroxylated by treatment using an excimer lamp, and finally amine-terminated by formation of a self-assembled silane monolayer with terminal amine functional groups (e.g., by deposition of a layer of 3-(aminopropyl)trimethoxy silane (“APTMS”) or 3-(aminopropyl)triethoxy silane (“APTES”).

Linker Moiety

Referring still to FIG. 1, in some embodiments, the electrochemical-based sensor according to the present disclosure further comprises a linker moiety that facilitates tethering a receptor, which is capable of selectively binding a target (e.g., biomolecule or analyte), to the solid electrode surface. The linker moiety may comprise any suitable chemical linking motif to provide secure attachment of the receptor molecules to the surface of the solid electrode material and may comprise any combination of organic, inorganic, biological, metallic, or other types of compounds. In some embodiments, the linker moiety comprises a biotin-streptavidin complex. In some embodiments, an amine-terminated BDD surface is biotinylated by carboxylic acid group activation chemistry using 1-ethyl-3-(3-dimehtylaminopropyl)carbodiimide (“EDC”)/N-hydroxysuccinimide (“NHS”).

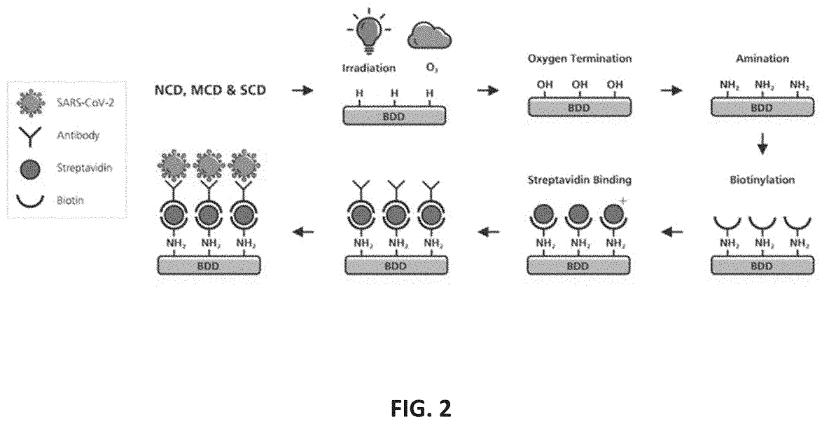

Referring to FIG. 2, in some embodiments, the sensor comprises a biotin-streptavidin linker complex to attach biotinylated antibodies to the surface of BDD. This is accomplished by first terminating the BDD with —NH₂ functional groups that can covalently bind the terminal —COOH group of biotin. Therefore, in some embodiments, any number of other linker molecules may be used, so long as there is an accessible —COOH group present to react with terminal —NH₂ groups on BDD. Further, in some embodiments, if streptavidin is omitted from the linker moiety, there may be no need to biotinylate the antibodies before attachment. Antibody attachment may be accomplished by reacting the terminal —NH₂ group of the antibody to a second —COOH group on the linker molecule. A non-exhaustive list of compounds that satisfy these criteria includes terephthalic acid, isophthalic acid, 2,6-naphthalenedicarboxylic acid, oxalic acid, malonic acid, succinic acid, glutaric acid, or other dicarboxylic acid compounds.

Receptor

In some embodiments, the electrochemical-based sensor according to the present disclosure comprises a receptor that is capable of selectively and specifically binding one or more particular targets (e.g., biomolecules or analytes). Referring to FIG. 2, in some embodiments, the receptor is any antibody or antibody fragment that specifically binds a target biomolecule of interest. In some embodiments, the receptor may be a biotinylated antibody or biotinylated antibody fragment that is bound to a streptavidin moiety present on the solid electrode material surface. In some embodiments, the receptor comprises an antibody specific for a pathogen (e.g., SARS-CoV-2). In some embodiments, the antibody is biotinylated. In some embodiments, the antibody is specific for a spike protein of SARS-CoV-2. In some embodiments, the antibody is SARS-CoV-2 (2019-nCoV) Spike S₁ Antibody, Rabbit MAb (Sino Biological; Cat: 40150-R007). In some embodiments, the antibody is specific for a S₁ subunit of the spike protein. In some embodiments, the antibody is specific for a S₂ subunit of the spike protein. In some embodiments, the target comprises SARS-CoV-2 (e.g., live SARS CoV-2).

Typically, an antibody consists of four polypeptides: two identical copies of a heavy (H) chain polypeptide and two copies of a light (L) chain polypeptide. Typically, each heavy chain contains one N-terminal variable (V_{H}) region and three C-terminal constant (C_{H}1, C_{H}2 and C_{H}3) regions, and each light chain contains one N-terminal variable (V_{L}) region and one C-terminal constant (C_{L}) region. The variable regions—which each comprise three complementarity determining regions (CDRs)—of each pair of light and heavy chains form the antigen binding site of an antibody.

The terms “antibody fragment” and “nicotine-binding fragment,” as used herein, refer to one or more portions of a nicotine-binding antibody that exhibits the ability to bind nicotine. Examples of binding fragments include (i) Fab fragments (monovalent fragments consisting of the V_{L}, V_{H}, C_{L} and C_{H1} domains); (ii) F(ab′)₂ fragments (bivalent fragment comprising two Fab fragments linked by a disulfide bridge at the hinge region); (iii) Fd fragments (comprising the V_{H} and C_{H1} domains); (iv) Fv fragments (comprising the V_{L} and V_{H} domains of a single arm of an antibody), (v) dAb fragments (comprising a V_{H} domain); and (vi) isolated complementarity determining regions (CDR), e.g., V_{H} CDR3. Other examples include single chain Fv (scFv) constructs. See e.g., Bird et al., Science, 242:423-26 (1988); Huston et al., Proc. Natl. Acad. Sci. USA, 85:5879-83 (1988). Other examples include nicotine-binding domain immunoglobulin fusion proteins comprising (i) a nicotine-binding domain polypeptide (such as a heavy chain variable region, a light chain variable region, or a heavy chain variable region fused to a light chain variable region via a linker peptide) fused to an immunoglobulin hinge region polypeptide, (ii) an immunoglobulin heavy chain C_{H2} constant region fused to the hinge region, and (iii) an immunoglobulin heavy chain C_{H3} constant region fused to the C_{H2} constant region, where the hinge region may be modified by replacing one or more cysteine residues with, for example, serine residues, to prevent dimerization. See, e.g., US 2003/0118592; US 2003/0133939.

In some embodiments, an antibody or antibody fragment as used herein may be IgG2, IgG3, IgA1, IgA2, IgE, IgH, or IgM, for example. In some embodiments, the antibody or antibody fragment may be mammalian, human, humanized, or chimeric.

In some embodiments, the receptor may specifically or selectively bind to a particular target (e.g., biomolecule or analyte). In some embodiments, the target may be a protein or protein subunit expressed on the surface of a pathogen (e.g., virus or bacteria). In some embodiments, the target may be the S₁ subunit or the S₂ subunit of the SARS-CoV-2 spike protein (also known as “S protein” or “glycoprotein S”) or a fragment thereof. The spike protein comprises two functional subunits responsible for binding to the host cell receptor (S₁ subunit) and fusion of the viral and cellular membranes (S₂ subunit). The SARS-CoV-2 spike protein (NCBI Reference Sequence: YP_009724390.1) comprises 1273 amino acids shown below:

(SEQ ID NO: 1)
MFVFLVLLPLVSSQCVNLTTRTQLPPAYTNSFTRGVYYPDKVFRSSVLHST
QDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWM
ESEFRVYSSANNCTFEYVSQPFLMDLEGKQGNFKNLREFVFKNIDGYFKIY
SKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSF
TVEKGIYQTSNFRVQPTESIVRFPNITNLCPFGEVFNATRFASVYAWNRKR
ISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYLYRLFRKS
NLKPFERDISTEIYQAGSTPCNGVEGFNCYFPLQSYGFQPTNGVGYQPYRV
VVLSFELLHAPATVCGPKKSTNLVKNKCVNFNFNGLTGTGVLTESNKKFLP
FQQFGRDIADTTDAVRDPQTLEILDITPCSFGGVSVITPGTNTSNQVAVLY
QDVNCTEVPVAIHADQLTPTWRVYSTGSNVFQTRAGCLIGAEHVNNSYECD
IPIGAGICASYQTQTNSPRRARSVASQSIIAYTMSLGAENSVAYSNNSIAI
PTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTD
EMIAQYTSALLAGTITSGWTFGAGAALQIPFAMQMAYRFNGIGVTQNVLYE
NQKLIANQFNSAIGKIQDSLSSTASALGKLQDVVNQNAQALNTLVKQLSSN
FGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQ
EKNFTTAPAICHDGKAHFPREGVFVSNGTHWFVTQRNFYEPQIITTDNTFV
SGNCDVVIGIVNNTVYDPLQPELDSFKEELDKYFKNHTSPDVDLGDISGIN
ASVVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT

In some embodiments, the target may be live SARS-CoV-2. In some embodiments, the target may be selected from any protein encoded by the genome of SARS-CoV-2, which corresponds to the nucleotide sequence of GenBank Accession No. NC 045512.2, and which is incorporated by reference in its entirety.

In some embodiments, binding of one or more targets (e.g., biomolecules or analytes) to one or more receptors generates an electrical impedance associated with the presence and concentration of the target. In some embodiments, the binding of one or more targets (e.g., biomolecules or analytes) to one or more receptors causes an increase in the charge transfer resistance of the solid electrode material. In some embodiments, the binding of one or more targets (e.g., biomolecules or analytes) to one or more receptors causes an increase in the charge transfer resistance of the solid electrode material of at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, or greater, or any range or value thereinbetween.

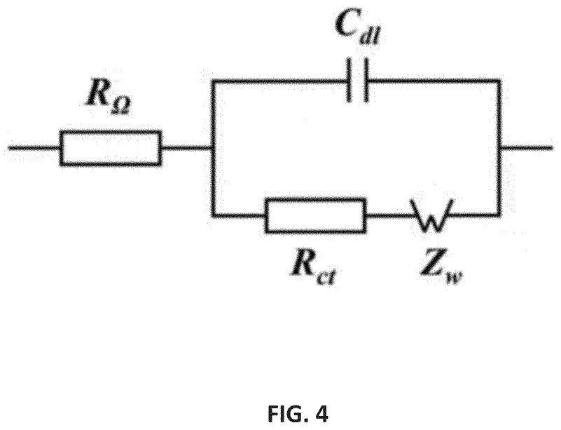

In some embodiments, the binding of one or more targets (e.g., biomolecules or analytes) to one or more receptors causes an increase in the charge transfer resistance of the solid electrode material that is detectable when the target concentration is no greater than 1 fg/mL, no greater than 2 fg/mL, no greater than 3 fg/mL, no greater than 4 fg/mL, no greater than 5 fg/mL, no greater than 6 fg/mL, no greater than 7 fg/mL, no greater than 8 fg/mL, no greater than 9 fg/mL, no greater than 10 fg/mL, no greater than 20 fg/mL, no greater than 30 fg/mL, no greater than 40 fg/mL, no greater than 50 fg/mL, no greater than 60 fg/mL, no greater than 70 fg/mL, no greater than 80 fg/mL, no greater than 90 fg/mL, no greater than 100 fg/mL, or greater, or any range or value thereinbetween. In some embodiments, the charge transfer resistance is measured using a conventional potentiostat equipped with EIS simulation software, and EIS spectra are fit to an appropriate circuit model (e.g., in FIG. 4), as discussed in the corresponding discussion in the Examples.

In some embodiments, the electrochemical-based sensor according to the present disclosure may comprise one or more than one type of receptor, such that a single sensor is capable of detecting one or multiple types of targets (e.g., biomolecules or analytes). In such “multi-modal” configurations, the binding of one or more than one type of target may create a unique electrochemical “fingerprint,” depending on the types of targets, their relative concentrations in the detection medium, their binding affinities for the one or more receptors, their binding kinetics with the one or more receptors, the relative concentrations of the one or more types of receptors within the sensor, locations of their respective receptors within the sensor, and environmental factors (e.g., media type, temperature, humidity, light conditions, etc.). In some embodiments, the digital fingerprint output caused by binding affinities for one or more receptors and targets is unique.

In some embodiments, electrochemical detection of a target biomolecule or analyte may use any suitable redox probe. By way of non-limiting example the redox probe may comprise potassium ferricyanide(III) (K₃Fe(CN)₆), hexaammineruthenium(III) chloride (Ru(NH₃)₆Cl₃), ferrocenecarboxylic acid (FcCOOH), ferrocenemethanol (FcMeOH), or any combination thereof

Reusable Sensors

The electrochemical-based sensors described herein may be a permanent, semi-permanent, disposable, or reusable component of a sensor system (e.g., including a housing, backend electronics, and a processor for analyzing sensor output). The solid electrode, linker moieties, and receptors may be capable of re-use or refurbishment by removing bound targets (e.g., biomolecule or analyte) via chemical and/or mechanical methods to re-expose receptors for binding targets (e.g., biomolecules or analytes).

Methods of Detecting a Target (e.g., SARS-CoV-2)

In some embodiments, the present disclosure relates to a method of detecting a target (e.g., analyte or biomolecule) in a medium, comprising: (a) contacting a sensor according to any of the embodiments discussed above with a medium containing or suspected of containing a target, wherein the contacting is performed over a time period during which the target present within the medium binds to one or more receptors on the sensor; (b) measuring a property (e.g., charge transfer resistance) of the sensor after the target has bound to the one or more receptors; (c) comparing the property (e.g., charge transfer resistance) of the sensor after the target has bound to the one or more receptors to the property (e.g., charge transfer resistance) of the sensor before the contacting to determine a change in the property of the sensor (e.g., an increase in charge transfer resistance); and (d) determining the amount of target present in the medium based on the change in the property of the sensor (e.g., the increase in charge transfer resistance). In some embodiments, the sensor comprises a solid electrode comprising boron-doped diamond (BDD).

In some embodiments, the property of the sensor measured to determine whether, and to what extent, a target has bound to the receptor is charge transfer resistance (R_{ct}). In some embodiments, the property may be a change in the peak current intensity for a redox probe from a square wave voltammetry measurement, e.g., as discussed in A. Chen & B. Shah, Electrochemical Sensing and Biosensing Based on Square Wave Voltammetry, 5 ANALYTICAL METHODS 2158-73 (2013), which is hereby incorporated by reference in its entirety.

In some embodiments, the medium may comprise a gaseous or liquid medium. In some embodiments, the medium comprises: ambient air, exhalation by a human or animal subject (e.g., as in a breathalyzer); a liquid comprising a physiological fluid (e.g., blood, plasma, serum, lymphatic fluid, cerebrospinal fluid, synovial fluid, urine, saliva, etc.), which may include a buffer (e.g., PBS); a municipal water source (e.g., from drinking water systems or wastewater systems associated with facilities, housing, or municipalities).

In some embodiments of the method, the target may be a protein or protein subunit expressed on the surface of a pathogen (e.g., virus or bacteria). In some embodiments, the target may be the S₁ subunit or the S₂ subunit of the SARS-CoV-2 spike protein (also known as “S protein” or “glycoprotein S”) or a fragment thereof. The spike protein comprises two functional subunits responsible for binding to the host cell receptor (S₁ subunit) and fusion of the viral and cellular membranes (S₂ subunit). The SARS-CoV-2 spike protein is described by NCBI Reference Sequence: YP 009724390.1. (See SEQ ID NO:1). In some embodiments, the method is a method of detecting SARS-CoV-2.

Sensor Networks

The ability of electrochemical-based sensors according to the present disclosure to rapidly and selectively detect one or more targets of interest opens up the possibility of rapid, selective, sensitive, and simultaneous detection of targets (e.g., biomolecules and/or analytes) at multiple locations to track the spread of pathogens or to track the progress of an outbreak. Such detection schemes could proceed by real-time testing of environments, surfaces, and environmental media.

Sensors and associated systems or networks according to the present disclosure may be designed for use associated with inanimate objects for detection of a target (e.g., biomolecule or analyte) in an environment (e.g., in air, in water, and/or on surfaces). For example, sensors and associated systems or networks may be installed in the ducts and/or filters of HVAC systems typically associated with large gatherings of people or nodal areas of passage (e.g., airplanes, schools, and shopping malls, subways, etc.) to facilitate continuous detection and tracing within large populations of individuals. In some embodiments, sensors located in HVAC systems or similar locations (e.g., to detect the presence and concentration of SARS-CoV-2 in the air) may further comprise an air sampling system to increase the air throughput (e.g., in instance when the concentration of SARS-CoV-2 is too dilute to permit detection without increased airflow). In some embodiments, sensors and associated systems or networks may be placed on surfaces and in/on/near HVAC systems to accumulate and detect airborne targets (e.g., SARS-CoV-2).

In some embodiments, sensors and associated systems or networks may be embedded in a water system (e.g., immersed in a water supply stream and/or in a wastewater stream) to detect the presence, concentration, and spread of targets (e.g. SARS-CoV-2) in water supply systems or wastewater systems associated with facilities, housing, or municipalities. In some embodiments, sensors and associated systems or networks according to the present disclosure may be embedded in or on frequently contacted surfaces (e.g., door handles, handrails, etc.) to facilitate surface screening for the presence of a target (e.g., SARS-CoV-2) in locations trafficked by large numbers of people (e.g., large entertainment or sporting event venues, mass transit facilities, and/or large office buildings).

Sensors and associated systems or networks according to the present disclosure may also be designed for human use to facilitate detection of a target (e.g., SARS-CoV-2) in human subjects in a wide range of scenarios where large numbers of human subjects are present or frequently travel. For instance, sensors and sensor networks may be used for security screening (e.g., at transportation facilities or entertainment venues), patient screening (e.g., at hospitals, at clinics, or in first responder scenarios).

Sensors and associated systems or networks according to the present disclosure also may be designed for personal/home use (e.g., in consumer products). In some embodiments, sensors for human use may be associate with an oral device, a breathalyzer, saliva collection, blood collection, or a swab-to-sensor configuration. In some embodiments, a disposable or reusable sensor may be associated with a processor (e.g., a device that interfaces with a mobile phone), such that a human subject may contact the sensor with a swab, saliva sample, blood sample, etc., then interface the sensor with a processor, which sends test data to a user interface (e.g., via a mobile app) for easy use and interpretation, followed by removal of the sensor from the processor for later disposal or reuse. In some embodiments, a sensor may be a disposable or reusable component of a face mask, embedded in a face mask, or as an integral part of face mask to detect data from the individual user's breath into said face mask, which then either transmits data to a central interface or the sensor is plugged into or otherwise coupled to (wired or wirelessly) a central interface to assess face mask users for interpretation.

Data extracted from sensor monitoring networks comprising one to thousands of sensors could be used by heath officials, environmental protection agents, homeland security agents, mass transit officials, and administrators to better react and respond to an outbreak, should an infected individual enter an area typically associated with large gatherings of people or areas which are highly-trafficked by large numbers of people, or should a target (e.g., SARS-CoV-2) be detected in a facility or system used by large numbers of people. This data could be used to inform effective policies, procedures, and methods of isolation and contact tracing in real-time, rather than requiring a multi-hour or multi-day detection methods that lag far behind the pace of an outbreak.

The sensor network may comprise a processor associated with each sensor. The processor may be configured to transmit, wired or wirelessly, to a central processor. The central processor can periodically request or continuously receive a status or data from each sensor. A sensor can also transmit upon detecting the presence of one or more targets (e.g., biomolecules or analytes).

Upon detecting the presence of one or more targets (e.g., biomolecules or analytes), the sensor can display an indicator, such as an LED light, or present on a display associated with the sensor. The sensor could also send a signal to a device (e.g., text message, email message) that alerts a user of the device to the positive result. The central processor can also display an indicator, present on a display, or transmit an alert to another device. The central processor can indicate which sensor of a plurality of sensors detected the one or more targets (e.g., biomolecules or analytes).

In another aspect, the present disclosure relates to a network of network of biomolecule sensors, comprising a first sensor according to the above disclosure, wherein the first sensor is placed at a first location; a second sensor according to the above disclosure, wherein the second sensor is placed at a second location; and a processing unit in communication with at least the first sensor and the second sensor. In some embodiments, the sensor may be used on humans (e.g., in an oral device, such as a breathalyzer), used on inanimate objects (e.g., embedded in an air filter, wastewater line, etc.) or applied in hybrid use applications (e.g. sensor integrated with a mobile phone). In some embodiments, the first location and/or the second location is associated with a heating and/or ventilation system, a surface (e.g., a contaminated surface), a breath-capture device (e.g., a breathalyzer), a sewer system, or a water supply system. A sensor network according to the present disclosure may comprise any suitable number of sensors, such as 1, 2, 3, 4, 5, 10, 20, 30, 40, 50, 100, 200, 300, 400, 500, 1,000, 10,000, or 100,000, or more sensors.

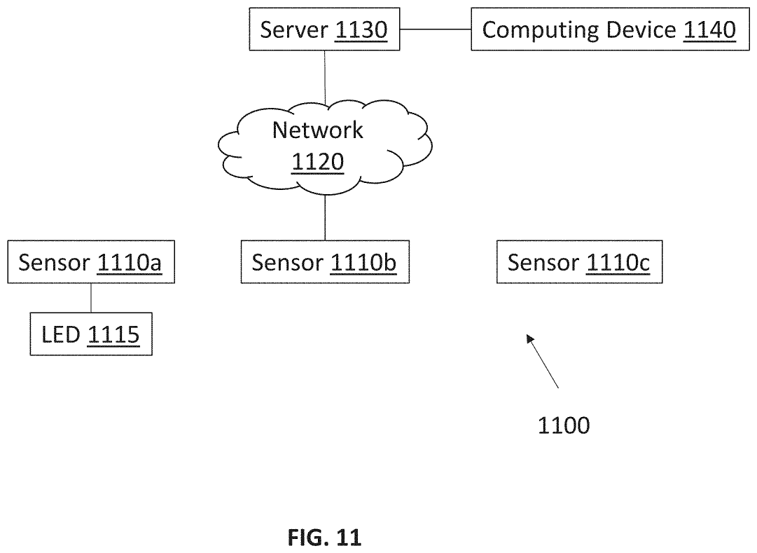

Referring to FIG. 11, a system architecture 1100 is shown according to an embodiment. System architecture 1110 includes a plurality of sensors 1110a, 1110b, 1110c. This illustration shows three sensors, though it is intended that only one sensor or more than three sensors may be utilized. Sensors 1110a, 1110b, 1110c may be positioned or integrated in similar or different environments, such as an HVAC system, entryway, home, handrail, arena, home, school, office building, mobile phone, breathalyzer, or other location.

The sensors 1110a, 1110b, 1110c may contain an indicator, such as an LED 1115, to show a positive detection. The indicator may be presented visually (such as LED 1115 or on a user interface) and/or audibly (such as a horn or siren).

The sensors 1110a, 1110b, 1110c may communicate via a network 1120 to a server 1130. The server 1130 may have a processing unit and a non-transitory computer-readable medium that stores instructions configured to be executed by the processing unit. A workstation or other computing device 1140 (e.g., laptop computer, mobile phone, tablet computer, desktop computer) may communicate with the server 1130 to display a dashboard of sensor status (e.g., active, inactive, positive detection, negative detection) as well as receive alerts and/or notifications.

The following examples are given for the purpose of illustrating various embodiments of the disclosure and are not meant to limit the present disclosure in any fashion. One skilled in the art will appreciate readily that the present disclosure is well adapted to carry out the objects and obtain the ends and advantages mentioned, as well as those objects, ends and advantages inherent herein. The present examples, along with the methods described herein are presently representative of embodiments and are exemplary, and are not intended as limitations on the scope of the disclosure. Changes therein and other uses which are encompassed within the spirit of the disclosure as defined by the scope of the claims will occur to those skilled in the art. It is to be understood that this present technology is not limited to particular methods, reagents, compounds compositions or biological systems, which can, of course, vary. The terminology used in the description herein is for the purpose of describing particular embodiments only and is not intended to be limiting.

### Examples

Electrochemical SARS-CoV-2 Sensor on BDD

BDD Surface Functionalization

Referring now to FIG. 2, electrochemical sensors for SARS-CoV-2 were prepared according to the procedure described herein below. Briefly, boron-doped diamond (“BDD”) was functionalized with antibodies for the S₁ subunit of the SARS-CoV-2 spike protein according to a procedure described in Rogien et al., “Surface Termination, Crystal Size, and Bonding-Site Density Effects on Diamond Biosensing Surfaces,” Diamond Rel. Mater. 106: 107843 (2020).

Hydrogen Termination

The BDD surface was cleaned via ultrasonication in acetone and methanol and/or by boiling in a 50:50 mixture of concentrated sulfuric acid and nitric acid. The samples were loaded in a MWPaCVD reactor and pumped down for 1 hour, then reacted with hydrogen plasma using 10 torr H₂, 200 sccm flow, and 1200 W microwave power for 10 min. The H termination was verified by measuring the contact angle of a water droplet.

Hydroxyl Termination

Freshly H-terminated BDD was cleaned using an ultrasonic bath of acetone, then methanol, and then rinsed with deionized water. The surface H groups were converted to hydroxyl (OH) groups by irradiating the samples with an excimer lamp at 172 nm for 1 hour. Successful OH termination was verified by measuring the contact angle of a water droplet.

Antibody Biotinylation

SARS-CoV-2 (2019-nCoV) Spike S₁ Antibodies, Rabbit Mab (hereinafter “anti-S₁”) (Sino Biological) were biotinylated to facilitate binding to the biotin-streptavidin complex on the BDD surface. An EZ-Link™ Micro Sulfo-NHS-Biotinylation Kit (ThermoFisher Scientific) and included protocol was used. A 1 mL volume of 10 μg/mL anti-S1 (stored at −20° C.) was thawed at room temperature. Sulfo-NHS-Biotin from the kit was diluted with 200 μL PBS, and the appropriate volume of the Sulfo-NHS-Biotin solution (according to the included protocol) was added to the thawed anti-S₁ solution. The reaction was incubated for 30-60 minutes. A Thermo Scientific Zeba Spin Desalting Column was prepared by breaking off the bottom plug and placing the column into a 15 mL collection tube. The column was centrifuged at 1000×g for 2 minutes, then the storage buffer was discarded and the column was returned to the same collection tube. A mark was placed on the side of the column where the compacted resin slanted upward. The column was placed in the centrifuge with the mark facing outward in all subsequent centrifugation steps. The column was equilibrated by adding 1 mL of PBS to the top of the resin bed and centrifuging at 1000×g for 2 minutes, discarding the flow through, then repeating this step 2-3 times. After being placed in a new 15 mL collection tube, the biotinylated anti-S₁ sample was added directly onto the center of the resin bed in the column and allowed to absorb. The column was centrifuged at 1000×g for 2 minutes, and the purified biotinylated anti-S₁ sample was collected in the flow through.

Antibody Attachment

Antibodies were attached to the hydroxylated BDD surface via a biotin-streptavidin linker complex. First, the surface OH groups were converted to amine (NH₂) groups by submerging the samples in a 30% (v/v) (3-Aminopropyl)trimethoxysilane (APTMS, 97%, Sigma-Aldrich) solution in methanol for 1 hour at room temperature. After this step, and after every subsequent step, the samples were washed 3 times in phosphate-buffered saline (PBS, pH=7.4, Sigma-Aldrich) for 1 minute on a gentle shaker (250 rpm).

The samples were placed in a cell-culture plate and immersed in 400 μL of 4 mg/mL biotin 99%, Sigma-Aldrich), 20 mg/mL 1-ethyl-3-(3-dimethylaminopropyl)carbodiimide hydrochloride (EDC, ThermoFisher Scientific), and 30 mg/mL N-Hydroxysuccinimide (NHS, 98%, Sigma-Aldrich) in PBS and incubated for 2 hours at room temperature, to covalently bind biotin to the surface NH₂ groups through its terminal carboxylic acid (COOH) group using standard COOH activation chemistry. To increase solubility, 1-4 drops of ammonium hydroxide solution (28% NH₃ in H₂O, ≥99.99%, Sigma-Aldrich) was added as needed. The samples were then immersed in 60 μL of 4 mg/mL streptavidin (Fisher Scientific) dissolved in PBS for 1 hour at room temperature. Lastly, to attach anti-S₁ antibodies, the samples were incubated in 400 μL of 4 μg/mL biotinylated (see biotinylation protocol above) SARS-CoV-2 (2019-nCoV) Spike S₁ Antibody, Rabbit MAb (Sino Biological; Cat: 40150-R007) in PBS overnight at 4° C. The functionalized surfaces were characterized by x-ray photoelectron spectroscopy (XPS) and atomic force microscopy (AFM) to verify the presence of N atoms and a thin film at the sensor surface (data not shown).

Electrochemical S₁ Detection

Electrochemical Cells and EIS Characterization

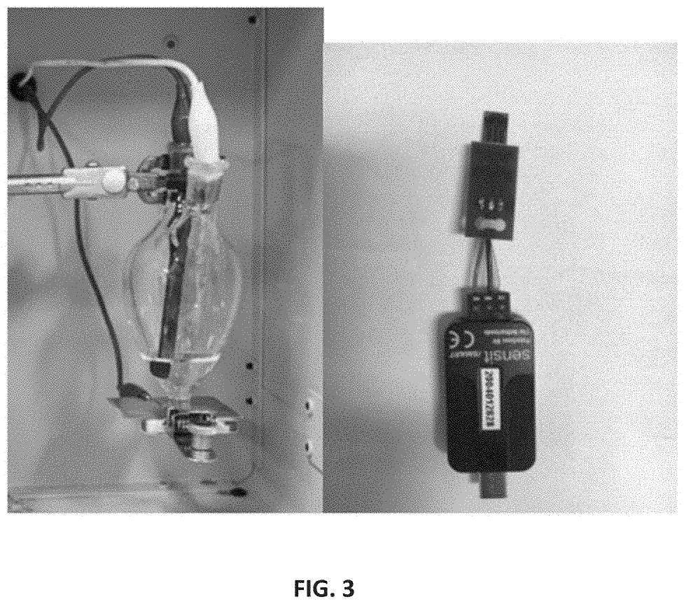

Referring now to FIG. 3 (left), functionalized 10×10 mm BDD samples were placed on the base of an electrochemical clamp cell, and a copper plate was clamped to the conductive silicon substrate to make the electrical connection to the potentiostat (CH Instruments, Inc.) through the working electrode lead. The active working electrode area was 3 mm in diameter (7.07×10⁻² cm² active surface area). An electrolyte solution containing 1 mM potassium ferricyanide(III) (K₃Fe(CN)₆, 99%, Sigma-Aldrich) in PBS was added to the cell. A graphite rod and platinum wire were used as the counter and reference electrodes, respectively. The potential of the Fe^{III/II} couple, E_{1/2}, where

$${E}_{\frac{1}{2}} = \frac{{E}_{p , a} + {E}_{p , c}}{2}$$

was measured using cyclic voltammetry (CV) scans between 0.8 V and −0.8 V. Controlled potential electrolysis (CPE) was used to perform a potential hold at E_{1/2} for 60 s to establish steady state conditions. EIS spectra were recorded using an E_{1/2} perturbation voltage, 10 mV AC signal amplitude, 100 kHz to 1 Hz frequency range. The resulting Nyquist plot was fit to the equivalent circuit model (FIG. 4) using the Levenberg-Marquardt method. The R_{ct} was obtained from the model fitting and plotted as a function of antigen concentration.

The working electrodes of 3-in-1 sensor chips with all BDD electrodes (working, counter and reference) was functionalized using the same procedure described above, but with volumes adjusted to accommodate the 2 mm diameter working electrode area. The switch to 3-in-1 sensors at this point was due to space limitations in the BSL3 lab as well as to provide a more user-friendly set up for the lab staff. The small sensor chips are capable of being connected to a palm-sized potentiostat (FIG. 3, right), as opposed to the bench top instrument used in all previous measurements. The working electrodes in this case are microelectrode arrays (MEAs) consisting of ˜97 individual BDD electrodes of 15 μm in diameter each, giving an active surface area of 1.71 ×10⁻⁴ cm². Using this setup, live virus binding measurements also may be conducted using an identical procedure as for S₁, though the incubation volume may need to be adjusted to account for any differences in working electrode area.

S₁ Incubation

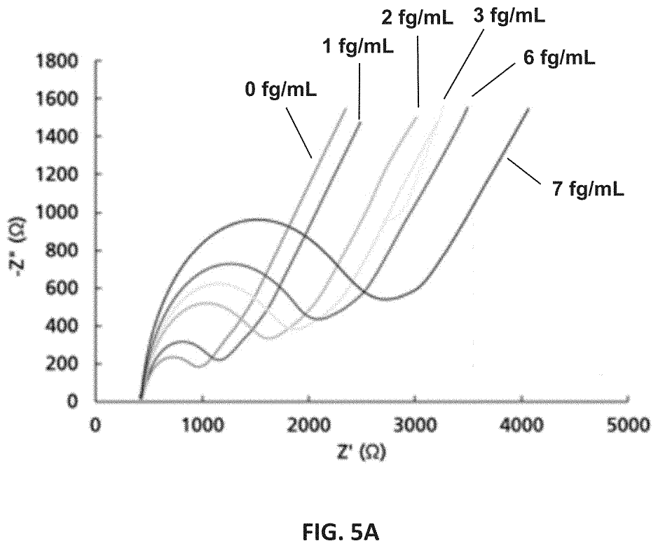

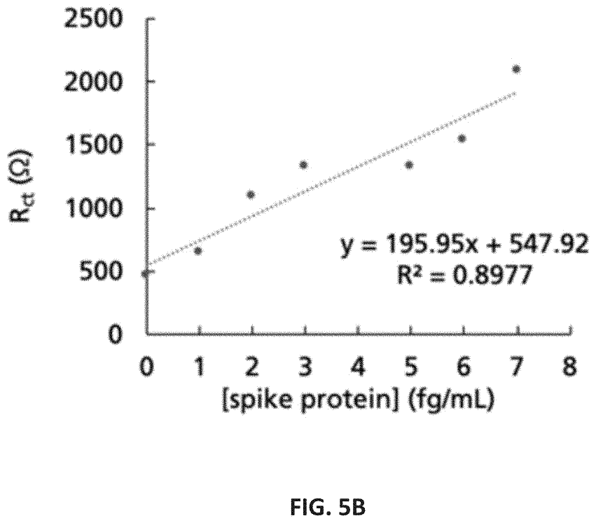

Referring now to FIG. 5A, binding of S₁ to the functionalized BDD surface was accomplished by incubating the sensor surface in 200 μL of solutions containing different concentrations of SARS-CoV-2 (2019-nCoV) Spike S₁-His Recombinant Protein (S₁, HPLC-verified, Sino Biological) in PBS for 5 minutes. After incubation, the surface was washed 3 times with PBS and the EIS spectrum was measured using the procedure described above. An identical protocol was followed for specificity testing with IFB (Influenza B [B/Brisbane/60/2008] Hemagglutinin Protein [HA1 Subunit, His Tag], Sino Biological).

Changes in charge transfer resistance (R_{ct}) were used to monitor binding of the spike protein S₁ subunit to the surface-bound antibodies. As shown in FIG. 5B, binding of the target protein resulted in an increase in R_{ct}, which was detectable at protein concentrations as low as 1 fg/mL. Target binding and detection were detected in as little as 30 seconds, indicating the rapid detection of SARS-CoV-2 afforded by this sensor architecture.

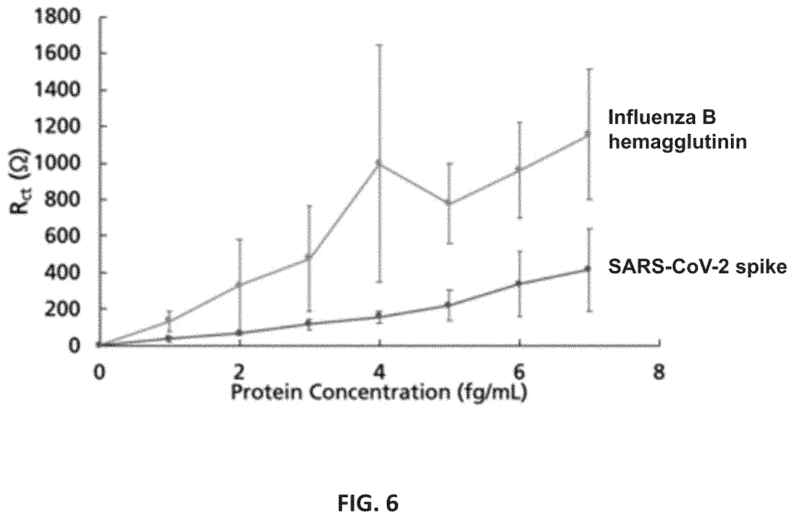

Referring now to FIG. 6, when tested against an Influenza B hemagglutinin protein, the sensor showed only minor fluctuations in R_{ct} that may be attributed to noise rather than specific binding, indicating that the sensor is specific for the SARS-CoV-2 spike protein.

Electrochemical BDD SARS-CoV-2 Sensors With Reduced Non-Specific Binding

Specific detection of SARS-CoV-2 in a complex matrix is challenging due to the increased number of non-specific interactions that can occur compared to detection in a simple PBS buffer. Thus, experiments were conducted to determine medium effects on the specificity of SARS-CoV-2 detection for BDD-based sensors prepared as described above.

In these experiments, M199 cell culture medium was used a model system to determine the ability of the sensors to detect SARS-CoV-2 in the presence of competing species. Early experiments showed that the typical detection method (incubation of electrode surface with sample, washing the surface, then adding electrolyte to perform EIS) resulted in large increases in R_{ct} from non-specific binding of components of the cell culture media. This made it difficult to distinguish between the signal resulting from specific binding of SARS-CoV-2 and the signal resulting from non-specific binding of other components in the cell culture medium.

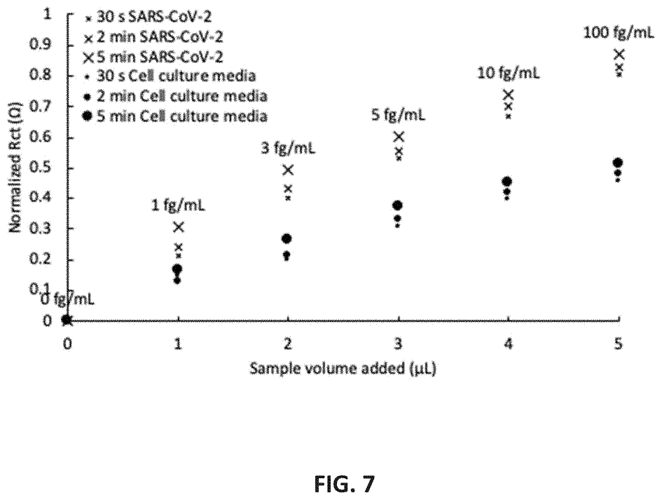

Detection of SARS-CoV-2 spike S₁ subunit on the functionalized BDD surface was accomplished by adding 1-μL aliquots of a range of SARS-CoV-2 (2019-nCoV) spike S₁-His Recombinant Protein (HPLC-verified, Sino Biological) concentrations in M199 cell culture medium (Sigma Aldrich) to 2 mL of an electrolyte mixture. The BDD surface was then incubated in the mixture for a minimum of 30 seconds prior to recording the EIS spectrum. The EIS spectrum was recorded before and after the sample additions, and increases in the R_{ct} due to specific binding of SARS-CoV-2 spike S₁ subunit were measured. FIG. 7 shows a comparison of the response to SARS-CoV-2 spike S₁ subunit versus the cell culture medium.

This method allows for continuous data collection so that the response over a range of sample incubation times could be measured. However, significant surface stabilization time was required when performing EIS measurements in this manner. Anywhere from 30 minutes to 120 minutes was required to establish a baseline R_{ct} value before adding the sample. Without being bound to any particular theory, this high stabilization time was likely due to the use of K₃Fe(CN)₆ as a redox probe. Though used extensively in impedimetric sensors, K₃Fe(CN)₆ has known stability issues, such as etching of gold surfaces and reactions with surface bound species. These R_{ct} stability issues did not arise in the initial measurements discussed above, in which sample incubation and EIS measurement occurred in separate steps, likely because the surface was not continuously exposed to the electrolyte mixture.

Thus, future studies will explore the relative stability and feasibility of other redox probes, including but not limited to hexaammineruthenium(III) chloride (Ru(NH₃)₆Cl₃), ferrocenecarboxylic acid (FcCOOH), and/or ferrocenemethanol (FcMeOH).

Effect of BDD Film Thickness on Specificity

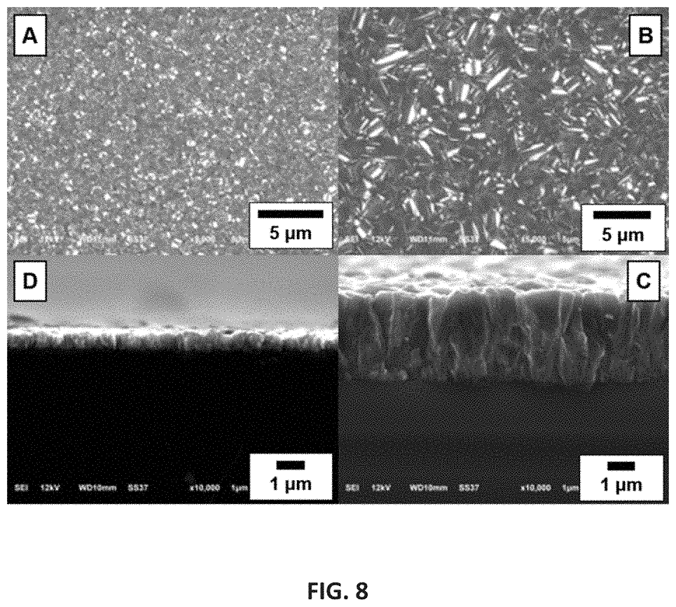

The effect of BDD material characteristics on detection response was further explored by varying the thickness of the BDD solid electrode material. A BDD film was obtained by using a shorter growth time, resulting in a thinner and smoother film, as determined by SEM analysis. Referring to FIG. 8, the BDD film thickness was 3.4 μm for the original sample (FIG. 8C) and 0.7 μm for the thinner film (FIG. 8D). As indicated in the top-down SEM images (FIGS. 8A-B), the thinner film (8A) has a smaller grain size than the thicker film (8B).

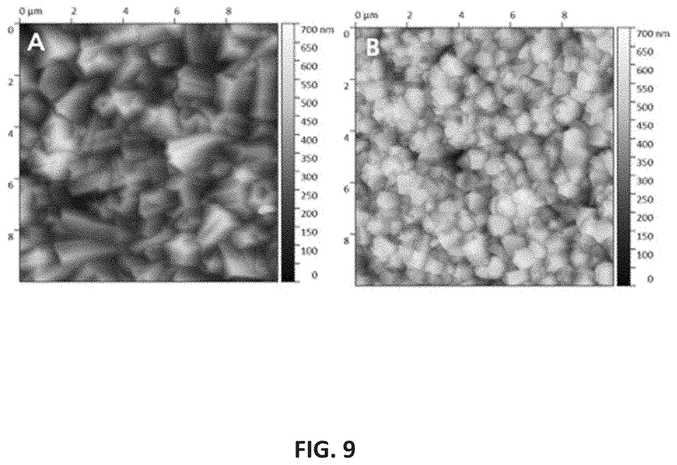

Atomic force microscopy (AFM; Hitachi 5000 II) was used to image the surface topography of each sample and to assess any changes in surface roughness with the addition of functional groups and biomolecules. A 10 μm×10 μm (512 px×512 px, 20 nm/px) area was scanned with a standard pyramidal AFM n-type silicon probe (MicroMasch®, HQ:NSC14/Al BS, tip radius 8 nm, resonance frequency 160 kHz, bulk resistivity 0.01-0.025 Ω·cm). The open source data analysis software Gwyddion was utilized to process the images and to determine the aerial root mean square surface roughness (S_{q}). FIG. 9 shows AFM images for the 0.7-μm (B, right) and 3.4-μm (A, left) BDD films. As shown in FIG. 9 (B), the thinner BDD film has smaller grains and a smoother surface than the original, thicker BDD film (A). The 0.7-μm and 3.4-μm BDD films show S_{q} values of 81.0 nm and 91.1 nm, respectively.

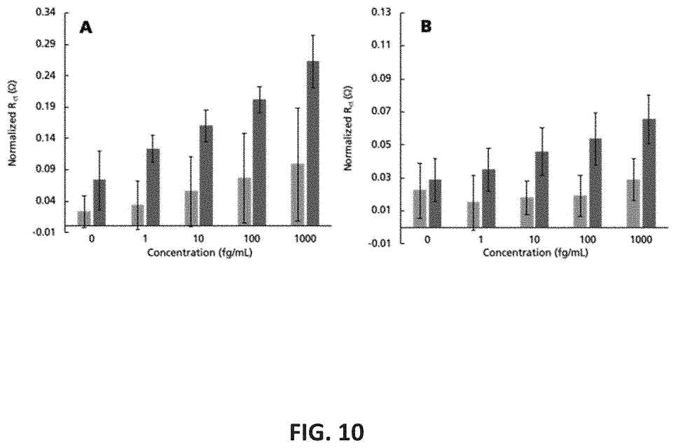

Based on initial results comparing both samples, it was hypothesized that a smoother surface would further increase the antibody loading density on the BDD surface and thereby enhance the electrochemical detection response. FIG. 10 shows the increase in R_{ct} as a function of S₁ subunit concentration for the both the thicker, 3.4-μm film (A, left) and the thinner, 0.7-μm film (B, right) using the detection method for reduced non-specific binding, discussed above, for samples suspended in cell culture media. Compared to the 3.4-μm film, the overall response to S₁ subunit binding was lower for the 0.7-μm film. However, when testing with Influenza B Hemagglutinin protein, the response was relatively flat for the 0.7-μm film compared to the thicker, 3.4-μm film, which exhibited an increase in R_{ct} with increased Influenza B Hemagglutinin protein concentration.

While not being bound to any particular theory, the decreased overall response of the thinner, 0.7-μm film to both proteins may indicate a decrease in antibody loading density and decrease of non-specific interactions with other matrix components. That is, a smoother film surface may decrease the amount of non-specific binding interactions of competing species, resulting in less contribution from interferents to the overall response. For both BDD films, the increase in R_{ct} at 0 fg/mL and 1000 fg/mL Influenza B Hemagglutinin protein are similar, indicating that it is possible to discern the response to S₁ subunit binding, even at high concentrations of interferents and relatively lower concentration of S₁ subunit.

Though the magnitude of the R_{ct} increase with increased antigen concentration is lower for the 0.7-μm BDD film, taken together, the data suggest that greater specificity can be achieved by using smoother BDD films.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 elements refers to groups having 1, 2, or 3 elements. Similarly, a group having 1-5 elements refers to groups having 1, 2, 3, 4, or 5 elements, and so forth.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All patents, patent applications, provisional applications, and publications referred to or cited herein are incorporated by reference in their entirety, including all figures and tables, to the extent they are not inconsistent with the explicit teachings of this specification.

What is claimed is:

1. An electrochemical-based sensor, comprising:
a solid electrode material;
a linker moiety bound to the solid electrode material; and
a receptor bound to the linker moiety,
wherein the receptor binds to a target, and the binding of target to receptor causes an increase in the charge transfer resistance of the sensor.

2. The sensor of claim 1, wherein the solid electrode material comprises boron-doped diamond.

3. The sensor of claim 1, wherein the linker moiety comprises a biotin-streptavidin complex.

4. The sensor of claim 1, wherein the receptor comprises an antibody specific for SARS-CoV-2.

5. The sensor of claim 4, wherein the antibody is biotinylated.

6. The sensor of claim 4, wherein the antibody is specific for a spike protein of SARS-CoV-2.

7. The sensor of claim 4, wherein the antibody is specific for a S₁ subunit of the spike protein.

8. The sensor of claim 4, wherein the antibody is specific for a S₂ subunit of the spike protein.

9. The sensor of claim 8, wherein the target comprises SARS-CoV-2.

10. The sensor of claim 1, wherein the target comprises live SARS-CoV-2.

11. The sensor of claim 1, wherein the sensor is reusable.

12. A network of biomolecule sensors, comprising:
a first sensor according to claim 1, wherein the first sensor is placed at a first location;
a second sensor according to claim 1, wherein the second sensor is placed at a second location; and
a processing unit in communication with the first sensor and the second sensor.

13. The network of biomolecule sensors of claim 12, wherein the first location is associated with a heating and/or ventilation system, water supply system, wastewater system, surface, breath capture device, saliva capture device, or nasal swab.

14. The network of biomolecule sensors of claim 12, wherein the second location is associated with a heating and/or ventilation system, water supply system, wastewater system, surface, breath capture device, saliva capture device, or nasal swab.

15. The network of biomolecule sensors of claim 12, wherein the first sensor and the second sensor are specific for the same target.

16. A method of detecting a target in a medium, the method comprising:
(a) contacting a sensor according claim 1 with a medium containing or suspected of containing a target, wherein the contacting is performed over a time period during which the target present within the medium binds to one or more receptors on the sensor;
(b) measuring a property of the sensor after the target has bound to the one or more receptors;
(c) comparing the property of the sensor after the target has bound to the one or more receptors to the property of the sensor before the contacting to determine a change in the property of the sensor; and
(d) determining the amount of target present in the medium based on the change in the property of the sensor.

17. The method of claim 16, wherein the target comprises SARS-CoV-2.

18. The method of claim 16, wherein the property is charge transfer resistance.

19. The method of claim 16, wherein the sensor comprises a solid electrode material comprising boron-doped diamond.

20. The method of claim 16, wherein the medium comprises at least one of: ambient air; exhalation by a human or animal subject; a liquid comprising a physiological fluid; a liquid from a municipal water source, or any combination thereof.


SEQUENCE LISTING (1 sequence)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT